(19) United States
(12) Patent Application Publication
(10) Pub. No.: US 2021/0251541 A1
(43) Pub. Date: Aug. 19, 2021

(54) EVALUATION OF A PERSON OR SYSTEM THROUGH MEASUREMENT OF PHYSIOLOGICAL DATA

(71) Applicant: EyeTracking LLC, Solana Beach, CA (US)

(72) Inventor: James J. Weatherhead, San Diego, CA (US)

(21) Appl. No.: 17/171,786

(22) Filed: Feb. 9, 2021

Related U.S. Application Data
(63) Continuation of application No. PCT/US21/17130.
(60) Provisional application No. 62/971,839, filed on Feb. 7, 2020.

(51) Int. Cl. A61B 5/16; A61B 5/00

(52) U.S. Cl. CPC A61B 5/165; A61B 2560/0223; A61B 2505/09; A61B 5/486

(57) ABSTRACT

Described and illustrated herein are methods and systems for the use of physiological data relating to cognitive state of a user, recorded from one or more sensors, for analyzing and reporting assessments of the cognitive state of the user.

### TECHNICAL FIELD

The subject matter described herein relates to approaches for use of physiological data recorded from one or more sensors either attached or remote stand-off from a person for analyzing and reporting assessments of one or more cognitive characteristics of the person as they correlate to the person's interaction with a system.

A person's interactions with various environmental factors can have differing impacts on that person's cognitive state. Certain situations can induce relatively small changes in cognitive state while other situations can cause much greater mental effort. Particularly for persons performing tasks that have health or safety impacts, such additional mental effort has the potential to jeopardize the user and/or others. As a particular example, a modern aircraft control system may have multiple screens, lights, indicators, buttons, controls, etc. for providing information to an operator and converting operator input to control operations for the aircraft. Current technologies are lacking in the ability to quantitatively discern whether one such system is more taxing on users than another. However such information can be quite important to have. While a typical user may have the ability to competently operate two systems with differing tendencies to induce changes in cognitive state while being operated, the less taxing system is desirable to avoid operator burnout, to preserve an operator's finite reserves of cognitive resources for periods of unexpected stress, etc.

### SUMMARY

Features of the current subject matter can provide benefits that address certain challenges inherent in assessing how “user friendly” a system is. The inventor has discovered, among other features described herein, that collection of data representative of a cognitive state of a user while that user interacts with a system can be used in conjunction with details of the user interaction to identify systems that induce more or less of a change in cognitive state to perform similar tasks. Such feedback can also be applied to identify particular users who may be having trouble with one or more aspects of such systems, to improve training systems (e.g., flight simulators or other vehicle driving or piloting simulators, augmented or virtual reality training programs, etc.), or the like.

In one aspect a method includes determining a cognitive state of a user in association with use of a system. The method further includes collecting data representative of a cognitive state of a user while the user performs one or more tasks while interacting with the system; concurrently recording details of the one or more tasks and actions of the user while performing the one or more tasks; and analyzing the data representative of the cognitive state and the recorded details of the one or more tasks and/or actions of the user, the analyzing including correlating the data with the recorded details to determine a metric representative of an amount of cognitive state change induced by the system in the user.

In embodiments, the method further includes repeating the collecting data and the concurrently recording details for a plurality of users interacting with the system; and generating a statistical measure of cognitive state induced by the system on a representative user. In embodiments, the method further includes comparing the statistical measure of cognitive state induced by the system with a second statistical measure of cognitive state induced by a second system; and ranking the first system as superior to the second system when the statistical measure of cognitive state induced by the system is lower than the second statistical measure of cognitive state induced by the second system. In embodiments, the method further includes comparing the metric representative of the amount of cognitive state change induced by the system in the user with the statistical measure of cognitive state induced by the system on the representative user; and identifying the user as a candidate for additional training when the metric representative of the amount of cognitive state change induced by the system in the user is higher than the statistical measure of cognitive state induced by the system on the representative user by a statistically significant threshold.

In another, interrelated aspect, a system for determining cognitive state of a user in association with a task is provided. The system includes one or more sensors configured to collect data representative of a cognitive state of a user while the user performs one or more tasks while interacting with the system; and a storage unit configured to concurrently record details of the one or more tasks and actions of the user while performing the one or more tasks; wherein analysis of the data representative of the cognitive state and the recorded details of the one or more tasks and actions of the user is performed, the analysis including correlating the data with the recorded details to determine a metric representative of an amount of cognitive state change induced by the system in the user.

In embodiments, the collecting data and the concurrently recording details for a plurality of users interacting with the system is repeated, and a statistical measure of cognitive state induced by the system on a representative user is generated. In embodiments, the statistical measure of cognitive state induced by the system with a second statistical measure of cognitive state induced by a second system is compared, and the first system is ranked as superior to the second system when the statistical measure of cognitive state induced by the system is lower than the second statistical measure of cognitive state induced by the second system. In embodiments, the metric representative of the amount of cognitive state change induced by the system in the user is compared with the statistical measure of cognitive state induced by the system on the representative user, and the user is identified as a candidate for additional training when the metric representative of the amount of cognitive state change induced by the system in the user is higher than the statistical measure of cognitive state induced by the system on the representative user by a statistical significant threshold.

In embodiments, the concurrently recording details of the one or more tasks and actions further includes temporally correlating the data representative of a cognitive state of the user with a specific task or action of the one or more tasks and actions.

In another, interrelated aspect, an apparatus for determining cognitive state of a user in association with a task, including one or more physiological sensors capable of obtaining data from the user and communicating the data obtained to an external device or apparatus, is provided. In embodiments, the communication is wireless. In embodiments, the communication occurs over a hard-wired connection.

In embodiments, the data representative of a cognitive state of a user or physiological data obtained by the sensor includes fatigue level, eye movement data, eyelid data, heart rate, respiration rate, electroencephalography (EEG) data, Galvanic Skin Response, functional near-infrared (fNIR) data, electromyography (EMG) data, head position data, head rotation data, emotion, excitement level, Facial Action Coding System (FACS) data, pupillometry, eye tracking data, or cognitive workload data.

In embodiments, the task includes a memory training task, a flight training task, a flight simulation task, a virtual surgical task, a virtual driving task, a cognitive assessment task, a cognitive aptitude task, command and control task, air-traffic control task, security monitoring task, vigilance task, a skill aptitude task, or a data entry task. In embodiments, the one or more sensors further include a calibration unit for calibrating the one or more sensors to the user. In embodiments, the calibration data is associated with the user based on a unique facial identification or fingerprint of the user, and stored for later recall. In embodiments, the cognitive state includes fatigue level, level of distress, level of excitation, emotion, anxiety level, cognitive overload, cognitive underload, distraction, confusion, level of boredom, a level of tunnel vision, a level of attention, level of stress, level of dementia, level of aptitude, or level of relaxation.

### BRIEF DESCRIPTION OF THE DRAWINGS

FIG. 1 is a diagram showing features of an example implementation of the current subject matter in which users interact with a first system to perform a task or tasks while data relating to cognitive state is collected for each user and with a second system to perform a task or tasks while data relating to cognitive state is collected for each user;

FIGS. 2A-2B are diagrams showing features of another example implementation of the current subject matter in which users interact with a system to perform a series of tasks while data relating to cognitive state is collected for each user during each task to enable identifying users having higher than expected changes in cognitive state and/or to identify tasks among a set of tasks that tend to induce higher than expected changes in cognitive state in one or more users while the users interact with the system and perform the tasks;

FIG. 3 is a diagram showing features of another example embodiment of the system disclosed herein in which a user interacts with a system to perform a series of tasks while data relating to cognitive state is collected for the user;

FIG. 4 is a diagram showing features of an example system consistent with the current disclosure; and

FIG. 5 is a process flow chart showing features of a method consistent with the current disclosure.

The details of one or more variations of the subject matter described herein are set forth in the description below. Other features and advantages of the subject matter described herein will be apparent from the description, and from the claims.

### DETAILED DESCRIPTION

The current disclosure relates to approaches for use of physiological data recorded from one or more sensors either attached to or remote stand-off from a person for analyzing and reporting assessments of one or more cognitive characteristics of the person, which may be useful for a range of applications. Such applications can include training pilots, drivers, surgeons, security personnel, command and control operators, air traffic controllers, etc. Such data may at first glance seem like it can only be used to learn about the person or persons that the system is monitoring. However, it has been discovered that monitoring how a given person or persons responds while using new systems or interfaces can provide useful information for learning about those systems and actually generate metrics for those systems based on the effect that those systems have on their users. In other words, the cognitive effects of a single system, or the comparative effects of two or more systems on a user or users interacting with such systems, can be quantified and used as a basis for identifying ways to modify or otherwise improve such systems for better usability, functionality, or the like. In some examples in which comparisons between systems are made, a selection of one system as better or otherwise more desirable that than another can be made based on comparison of one or more metrics of cognitive state changes induced in user so the two systems. In other examples, such a metric of cognitive state change can be used in identifying one or users out of a larger cohort of users who are experiencing a more pronounced cognitive state change relative to some statistical measure (e.g., an average, a median, some deviation from an average, a threshold, discriminant function analysis, a neural network, or other advanced statistically based classification techniques, etc.) of the larger cohort.

One example consistent with the current disclosure could relate to a new system being created for an aircraft. If the manufacturer wishes to know whether the new system requires more or less mental or cognitive effort to operate than the current system in use, the systems and methods consistent with the current subject matter are capable of collecting cognitive state and/or eye tracking data of one or more operators (e.g., users) as they use the two versions of the system for the aircraft (i.e., a current system vs a new or updated system) and providing objective feedback about the experience that the two interfaces impose on their users. For example, it can be possible to determine and quantitatively or qualitatively report whether the user workload is higher on one platform versus another (thereby showing that it is more difficult). In the example of a pilot flying an aircraft, a pilot may land a plane many times well using two different systems, but using the systems and methods herein one may detect a higher workload for the pilot when using one platform versus another. Although both platforms may allow completion of a task, it can be determined that a user is required to put in more mental effort when using one platform and may therefore become mentally fatigued more quickly, may be more prone to error, and may have less mental capacity to deal with other critical tasks that may be expected of him or her.

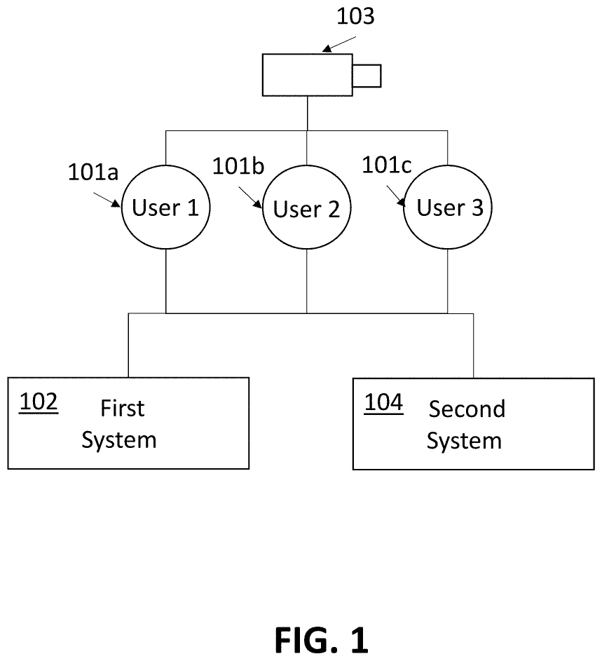

FIG. 1 is a block diagram illustrating some features of an example embodiment of the current disclosure. A plurality of users 101a-101c interact with a first system 102. As the users 101a-101c interact with the system 102, data representative of a cognitive state (which, as used herein can optionally include either or both of data directly indicative of a user's cognitive state or data from which a measure of a user's cognitive state or a proxy thereof can otherwise be estimated or calculated) of each user 101a-101c is collected by one or more measurement devices 103. These data can include physiological data and/or other data, including but not limited to one or more of pupillometry data, fatigue level data, eye movement or eye tracking data, eyelid data, heart rate data, respiration rate data, electroencephalography (EEG) data, Galvanic Skin Response data, functional near-infrared (fNIR) data, electromyography (EMG) data, head position data, measures of emotion, excitement level, Facial Action Coding System (FACS) data, and cognitive workload data. In some examples, the data representative of a cognitive state can be or include eye movement and eye scan data as such measurable characteristics can be highly indicative of a user's effort required to perform tasks presented on a screen or other user interface, display, etc.

During the performance of one or more tasks by a user as that user interacts with the system 102, details of those one or more tasks are concurrently recorded (e.g., along with the collection of the data representative of user cognitive state). The types of detail recorded can include, without limitation, one or more of a start and/or stop time of the user's interaction with the system, a start and/or stop time of one or more sub-tasks or events or actions occurring or performed by the user during the user's interaction, specific regions of a screen or other user interface element that were activated or otherwise displaying information or requesting user input, other factors such as auditory or haptic alerts provided to the user, etc. The data representative of the cognitive state and the recorded details of the one or more tasks and actions of the user or users can be analyzed, which can include correlating the data with the recorded details to determine a metric representative of an amount of cognitive state change induced by the first system in a given user or users.

In the example of FIG. 1, the users 101a-101c (who may optionally be other users than those who interacted with the first system 102) may interact with a second system 104 (e.g., at some time before or after the interaction with the first system 102) while the same or similar data representative of a cognitive state is collected by one or more devices 103 (which can be the same one or more devices 103 as are used with the first system or some different one or more devices 103). As with the users' interaction with the first system 102, details of the one or more tasks are concurrently recorded, and the data representative of the cognitive state and the recorded details of the one or more tasks and actions of the user or users can be analyzed, which can include correlating the data with the recorded details to determine a metric representative of an amount of cognitive state change induced by the second system in a given user or users. A comparison can then be made between the respective metrics representative of an amount of cognitive state change of the first and second system. Such a comparison can be useful in assessing which of the first system 102 or the second system 104 induces greater cognitive effort in users. Analysis of particular features of the systems can be performed, for example to identify one or more specific aspects of user interaction with a system that lead to increased changes in cognitive state on users (e.g., such as greater fatigue, greater eye movements, distractions, etc.). Using this analysis, which can be performed at any temporal granularity supported by the collected data and recorded details, specific tasks of a single system may be identified as needing better workflow or otherwise being made less complicated, etc. to improve changes in user cognitive state during their execution.

Additionally, another exemplary use of the systems and methods describe herein can allow for measurement of the expertise of a user and to provide feedback as to how the user is progressing through training against both their own benchmark performances and/or relative to one or more peers, using objective data. For example, detection of a high change in cognitive state in a user may be indicative of a flaw in a training process, some part of the user's interaction with or approach to handling information provided by a system, etc. that need improvement. Alternatively, such measures could be used to identify a user who is not performing at optimum levels on a given day, which can signal that that user may need to be replaced or assisted with critical tasks.

In a training scenario, if a user's cognitive workload (e.g., change in cognitive state) does not show signs of decreasing after repeated practice, the user may be flagged for more training, and may be indicated as not improving as well as expected in a training process. This can prompt a trainer to investigate the underlying reasons and to modify the training process accordingly. For example, indications that a user being trained on a flight simulator is having higher than expected cognitive state changes could trigger a trainer to determine (possibly by using concurrent eye-tracking data for the user during the training process) that the user is not utilizing an optimal visual scan path across screen or visual indicators in an aircraft cockpit. Using approaches consistent with those described herein, it can also be possible to demonstrate whether a user misses key information on one system vs the other. For example, data may include quantifying whether a user is required to make more frantic, hence potentially less efficient, eye movements on one platform versus another, and so on.

Further to this example, data representative of a cognitive state (or data from which a measure of a cognitive state or a proxy thereof can otherwise be estimated or calculated) may be collected while one or more users (e.g., trainees) interact with a simulation system, for example one involving complex tasks such as flying an aircraft or spacecraft; driving a car, a train, truck, military tank or armored vehicle; piloting a ship or other marine vessel; performing robotic or laparoscopic surgery; performing one or more tasks such as command and control tasks, security monitoring tasks, or air-traffic control tasks; or the like. In such scenarios, the systems and methods described herein are capable of providing real-time feedback to experienced trainers or instructors. In some examples, the trainer can be in the room with the trainee so that the trainer can see exactly where the trainee is looking, what the trainee workload is, and any other physiological data relating to the trainee performing the task. Using the systems and methods described herein, one may also show the current cognitive state, the regions that have been identified, and any associated data such as how many times those regions have been viewed, what the cognitive state of the trainee was while the regions were being viewed, etc. Data can be pooled, both in real-time and/or off-line, to allow a user to find and illustrate trends in the data, such as for example to see a person's progression over time. For example, it may be desirable to have a trainee perform the same or comparable tasks over time. By using the systems and methods described herein, it can be possible to observe that trainee's eye scan paths changing over time, as well as whether their changes in cognitive state trend in a desired direction resulting from use of the system over time. It can then be determined whether person is learning at an acceptable rate compared to their peers, or if retraining is suggested for a given task or tasks.

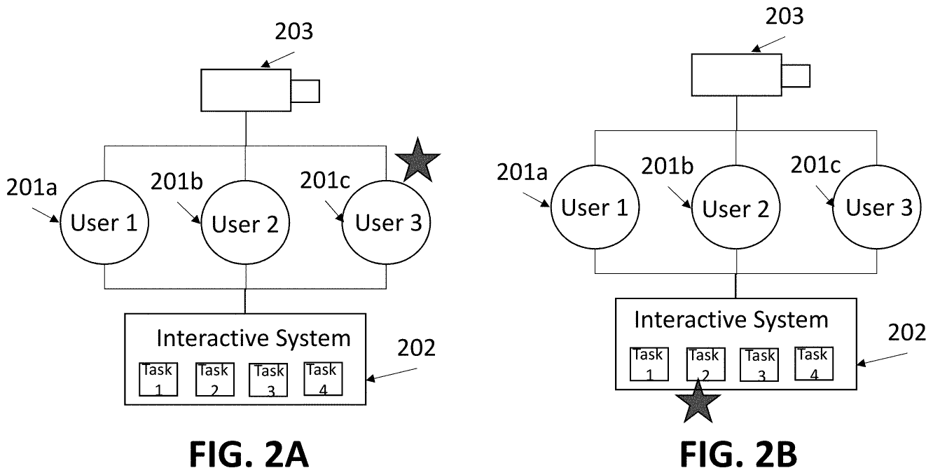

FIGS. 2A-2B show a depiction of an implementation consistent with this embodiment. As shown, a group of users 201a-201c interact with a system 202, where the user interactions including performing one or more tasks. As a user 201a-201c interacts with the system 202, data representative of a cognitive state of the user 201a-201c is collected by a device or sensor 203, and details of the one or more tasks and actions of the user while performing the one or more tasks are concurrently recorded. As shown in FIG. 2A, a user 201c can be flagged if they exhibit a cognitive state that deviates from an expected cognitive state, for example, if a higher change in cognitive state is indicated by the collected data. As shown in FIG. 2B, a task among multiple tasks involved in user interaction with the system 202 can be flagged if analysis of the collected data and the recorded details indicates that the task induces a cognitive state in the users 201a-201c that deviates from an expected cognitive state.

Another example consistent with the current disclosure relates to research into medical diagnosis applications, such as for example when researching dementia. The software described herein can allow for recording of a series of eye movements, workload, and/or other physiological data or cognitive state data generated as a person performs a series of benchmark tests. The software can then report changes in workload and/or eye scan patterns over time, and/or other relevant metrics including cognitive state data. Statistical tests can then be created and incorporated into the system to allow automated feedback as to the diagnosis or prognosis of a person having dementia on a task or series of tasks.

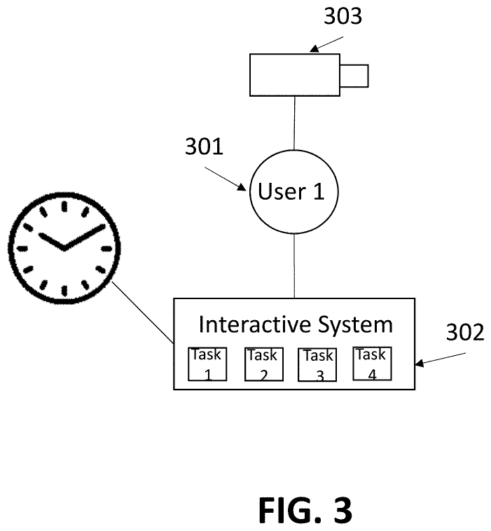

FIG. 3 illustrates another example embodiment in which a user 301 interacts with a system 302 to perform multiple tasks. As the user 301 interacts with the system 302, data representative of a cognitive state of a user 301 is collected by a device or sensor 303. Additionally, time data with correlated details about the user interactions (e.g., the user's completion of the tasks) with the system 302 is collected. A baseline measure of cognitive state, either for the whole of the interaction or broken up more granularly to correlate with the collected details can be created, and deviations from the baseline can be monitored as the user 301 interacts with the interactive system 302 on subsequent occasions.

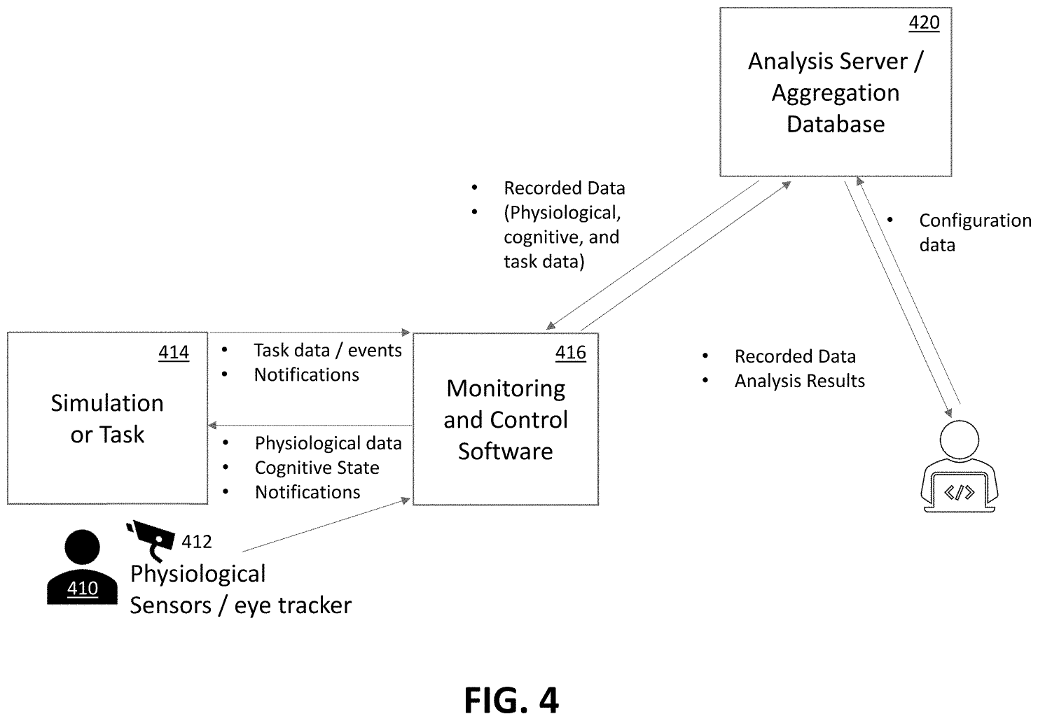

FIG. 4 shows a diagram illustrating example features of a system upon which features of the current disclosure may be implemented. Other systems are also within the current scope, and the features in FIG. 4 are intended merely as an illustrative depiction. As shown in FIG. 4, a user 410 is monitored by one or more physiological and/or eye-tracking sensors 412, from which data representative of a cognitive state of the user can be collected. The user 410 performs a simulation or task 414 (or multiple iterations or variations of same) while data and/or details about tasks, events, displayed notifications, etc. are collected. In this example, monitoring and control software (which can optionally be or include eye tracking software) 416 can receive the details about the task data/events and any notifications or other system interactions with the user 410. The monitoring and control software 416 can also optionally provide feedbacks to the system, such as prompting specific actions, alerts, notifications, etc. that are intended to elicit a user response. For example, the system can cause a warning to be displayed to gauge user reactions, whether the warning is detected and how quickly, what impact the warning has on the user's cognitive state, etc.

The monitoring and control software 416, which can be implemented on one or more processors, can optionally communicate with an analysis server or other aggregation database 420 that collects and analyses or enables analysis of recorded data representative of user cognitive state during interaction with the system as well as details about the one or more tasks being performed.

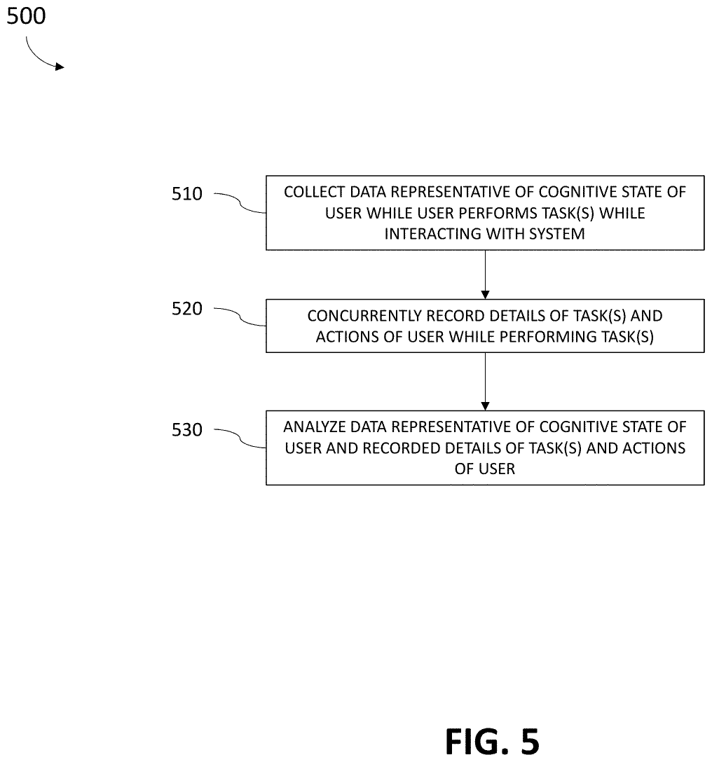

FIG. 5 shows a method flow chart 500 illustrating features consistent with one or more embodiments of the current disclosure. A method for determining cognitive state of a user in association with use of a system can be provided. Such a method can beneficially be supported by software running on one or more processors, and through the use of a system, which can include one or more sensors or devices for measuring or otherwise collecting data representative of a cognitive state of a user. Such data can include, but are not limited to one or more of fatigue level, eye movement data, heart rate, respiration rate, electroencephalography (EEG) data, Galvanic Skin Response, functional near-infrared (fNIR) data, electromyography (EMG) data, head position data, emotion, excitement level, Facial Action Coding System (FACS) data, pupillometry data, eye tracking data, eyelid data, or cognitive workload data.

At 510, data representative of a cognitive state of a user are collected while the user performs one or more tasks while interacting with a system. The one or more tasks can optionally include a memory training task, a flight training task, a flight simulation task, a virtual surgical task, a virtual driving task, a cognitive assessment task, a cognitive aptitude task a command and control task, an air-traffic control task, a security monitoring task, or a data entry task. The cognitive state can include one or more of the cognitive states comprising fatigue level, level of distress, level of excitation, emotion, anxiety level, cognitive overload, cognitive underload, distraction, confusion, a level of relaxation, level of boredom, level of attention, level of tunnel vision, or the like.

At 520, details of the one or more tasks and actions of the user are concurrently recorded while performing the one or more tasks. The concurrently recording details of the one or more tasks and actions further can optionally include temporally correlating the data representative of a cognitive state of the user with a specific task or action of the one or more tasks and actions.

The data representative of the cognitive state and the recorded details of the one or more tasks and actions of the user are analyzed at 530. This analyzing includes correlating the data with the recorded details to determine a metric representative of an amount of cognitive state change induced by the system in the user.

In optional variations, the collecting data and the concurrently recording details can be repeated or otherwise replicated for a plurality of users interacting with the system, and a statistical measure of change in cognitive state induced by the system on a representative user can be generated. This statistical measure of cognitive state change induced by the system can optionally be compared with a second statistical measure of cognitive state change induced by a second system or alternatively the statistical measure can be compared with a baseline or threshold metric to determine whether the first system needs to be improved in one way or another to reduce, for example, cognitive workload requirements on its users.

The systems and methods described herein provide an end-to-end data collection and automated analysis capability. These systems and methods can record cognitive state data from one or more eye trackers or other sensors receiving information from a person performing a task. The eye tracking data can include, but is not limited to: eye position, eye rotation, pupil size, object being looked at, area on object being viewed, blink rate, blink count, blink speed, amount of eye lid opening or eye lid closing, etc. Such data can be collected by a variety of devices, including in some instances visualization means, such as a camera or a specialized eye tracker or sensor that operates under visible light, or light in the infrared or near-infrared spectrum, to receive a visual image of the person which is then processed to generate the data. The system may also record one or more other types of physiological data such as, for example, electroencephalography (EEG) data, Galvanic Skin Response, functional near-infrared (fNIR) data, electromyography (EMG) data, electrocardiogram (ECG/EKG), galvanic skin response (GSR), respiration, head position, head rotation, facial expression, facial action coding system (FACS) data, emotion, stress level, fatigue level, level of arousal, and/or level of excitement. The system can then compute a measure of the cognitive state change of the person performing the task. Any combination of task, system, physiological, and/or behavioral data can be captured and/or processed in real time. Additionally and/or alternatively, data can be stored locally and/or be distributed to local, remote or cloud-based server, for real time or offline storage and/or processing. Data points can have events associated with them, or associated to the same time scale of the data points, which can indicate a moment in time, and the events can have a name, a description, a saliency, and/or a rating associated with them. In some examples, both tasks and events can be created by any of the following: a person being observed and/or viewed, a secondary person operating the system such as a trainer, or a separate computer system operating automatically such as a flight simulator or task generator or simulator. The systems described herein with which users interact can be, for example, virtual reality systems, augmented reality systems, mixed reality systems, headsets, interaction with a machine such as a vehicle or aircraft, or interaction with a building.

In cases where calibration data is needed for a particular use, the systems and methods described herein are capable of allowing the capture of calibration data and the recall of that calibration data at a time point in the future, such that re-calibration may not be required. This can save startup time in future uses. For example, some eye tracking systems must be calibrated prior to use. Using the systems and methods described herein, one may capture this information and associate it with a person, such that the next time the person uses the system, the calibration may be automatically recalled. The calibration may be collected, either on the disclosed system directly or on a separate system configured to capture calibration data, and then imported into the disclosed system manually and/or automatically.

Additionally, regions or areas of interest can be defined in the disclosed system to allow analysis of data collected, such as for example, scenarios in which the task being performed by a person is to perform a training task in a simulated environment, such as a flight simulator. In such a scenario, one may wish to define a first region to represent a primary flight display, and define a second region to represent a secondary flight display. The information used can be, for example, a map, a fuel gauge, a store level, etc. Once a region or regions have been defined, the system is able to perform computations including, but not limited to: how often each region is being viewed, the duration of time since a region was last viewed, the average duration of each viewing, the frequency of viewing, and/or the order in which regions were viewed.

Additionally, secondary physiological signals and/or task or performance data can be correlated with eye tracking data and defined regions (e.g., in a screen or other display that are part of the system with which a user interacts), to calculate the level of cognitive workload and/or the emotional state of a person that occurred while the person viewed each given region, on a case by case basis. Data relating to fatigue level or cognitive state, obtained using other sensors, can also be used to calculate the cognitive workload and/or emotional state of a person and associated with performing various tasks.

Regions may be static, or they may be dynamic. For example, a region may be defined to represent a target that moves around on a screen that a person is asked to monitor. In examples where a person is flying an aircraft, a region may be defined that tracks a secondary airport out of the aircraft's cockpit window. Whether the regions are static or dynamic, the regions can be analyzed in a similar manner. Dynamic regions may be defined in the software such that a pre-programmed path is followed by the region. Additionally and/or alternatively, dynamic regions may be controlled by algorithms to follow a visual object on a screen or in a video feed. Dynamic regions may be controlled by user inputted key-frames and interpolated over time. Dynamic regions may be controlled by a third party software as to their appearance over time. Dynamic regions may be generated by computer vision software using traditional pattern matching (e.g. Histogram of Oriented Gradients (HOG) filters and the like), or more modern approaches such as neural networks or similar. Dynamic regions may be controlled by non-visual sensors such as Light Detection And Ranging (LIDAR), ultrasonic sensors or the like. Dynamic regions may change size, shape, and/or visibility over time. Dynamic regions may behave differently from person to person.

While not explicitly required by the system, rules can be used. If used, rules can be standalone, or can be associated with regions. Rules can be used to signal alerts or events, or to trigger actions if a rule trigger is met. For example, one rule might be that a person must look at a specific region every 30 seconds. If the person fails to look at the specified region in the allotted time frame, a notification or alert can be sent to the system for further action. Rules can use a straight IF/THEN type logic, or rules can use a linear or non-linear algorithm (such as, for example, a neural network) to determine whether a rule is triggered. Additionally and/or alternatively, rules can be triggered by non-eye tracking data such as stress measurements, fatigue levels detected, etc.

Each recording or analysis can have a minimum of a subject identifier (such as name, employee record, or random id, for example) as a way to identify the task being performed (manual or automated). These markers may be explicit or implied. For example, if only one person who flies the same simulator in the same way each time they use the system is ever tracked, the marker is implied. In some examples, a user operator may enter the task or the subject prior to starting the system. Additionally and/or alternatively, the system may use facial identification, Near Field Communication (NFC), or fingerprint identification to identity a subject.

Analysis can be performed in real-time and/or offline. The system can save settings used in a current recording, which may be used when analyzing results as well as to force those settings onto the next recording to save time. The system can distribute saved settings or predefined settings to the software, prior to a new recording. The system can record video from zero, one, or more than one source and correlate eye-related data and other physiological data using the zero, one, or more than one source. Audio data can also be recorded, with or without accompanying video, and can be correlated to a video.

The system and methods described herein can allow for definition of one or more reports. The reports can be delivered textually, graphically or in a tabular format. Reports and the analysis behind the reports can be added, removed, or updated easily. As the data in the system grows, new findings can allow for new analysis and new reports to be built. Typically, these reports use statistical models (both linear and non-linear, such as deep learning models) to identify a finding. Findings can include information such as, but not limited to: which of multiple interfaces is easier to use than another, which trainees are ‘trained’ or ‘expert’ versus not, whether trainees are learning at a suitable rate, which of multiple tests is more difficult than another, etc. The analysis and reports may further include training and system evaluation into medical diagnostics, uses in Unmanned Aerial Vehicles (UAV), uses in command and control, uses in aircraft, uses in surgery, uses in air-traffic control, and more.

Any element of the above can be packaged into one software, or can be spliced into one or more remote components that work together to form the same or similar functions. Additionally and/or alternatively, the software can be used to report on one or more person's physiological behavior on one occasion, or over time. For example, the software can be used in diagnostic or certification purposes including but not limited to: reporting whether someone is cognitively impaired, reporting whether someone needs more training in one or more tasks or competencies, reporting whether someone is deemed trained or expert in some competency, reporting whether someone has dementia or some other cognitive impairment, reporting whether a piece of software results in a higher or lower workload than another, and/or reporting a training score or score(s).

Implementations of the current subject matter can include, but are not limited to, methods consistent with the descriptions provided above as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claim.

1. A method of determining cognitive state of a user in association with use of a system, the method comprising:
collecting data representative of a cognitive state of a user while the user performs one or more tasks while interacting with the system;
concurrently recording details of the one or more tasks and actions of the user while performing the one or more tasks;
analyzing the data representative of the cognitive state and the recorded details of the one or more tasks and/or actions of the user, the analyzing comprising correlating the data with the recorded details to determine a metric representative of an amount of cognitive state change induced by the system in the user.

2. The method of claim 1, further comprising:
repeating the collecting data and the concurrently recording details for a plurality of users interacting with the system; and
generating a statistical measure of cognitive state induced by the system on a representative user.

3. The method of claim 2, further comprising:
comparing the statistical measure of cognitive state induced by the system with a second statistical measure of cognitive state induced by a second system; and
ranking the first system as superior to the second system when the statistical measure of cognitive state induced by the system is lower than the second statistical measure of cognitive state induced by the second system.

4. The method of claim 3, further comprising:
comparing the metric representative of the amount of cognitive state change induced by the system in the user with the statistical measure of cognitive state induced by the system on the representative user; and
identifying the user as a candidate for additional training when the metric representative of the amount of cognitive state change induced by the system in the user is higher than the statistical measure of cognitive state induced by the system on the representative user by a statistically significant threshold.

5. The method of claim 1, wherein the concurrently recording details of the one or more tasks and actions further comprises temporally correlating the data representative of a cognitive state of the user with a specific task or action of the one or more tasks and actions.

6. The method of claim 1, wherein the data representative of a cognitive state of a user comprises fatigue level, eye movement data, eyelid data, heart rate, respiration rate, electroencephalography (EEG) data, Galvanic Skin Response, functional near-infrared (fNIR) data, electromyography (EMG) data, head position data, head rotation data, electrocardiogram (ECG/EKG) data, emotion, excitement level, Facial Action Coding System (FACS) data, pupillometry, eye tracking data, or cognitive workload data.

7. The method of claim 1, wherein the one or more tasks comprise a memory training task, a flight training task, a flight simulation task, a virtual surgical task, a virtual driving task, a cognitive assessment task, a cognitive aptitude task, a command and control task, an air-traffic control task, a security monitoring task, a vigilance task, a skill aptitude task, or a data entry task.

8. The method of claim 1, wherein the cognitive state comprises one or more of fatigue level, level of distress, level of excitation, emotion, anxiety level, cognitive overload, cognitive underload, distraction, confusion, level of boredom, a level of tunnel vision, a level of attention, level of stress, level of dementia, level of aptitude, or level of relaxation.

9. A system for determining cognitive state of a user in association with a task, the system comprising:
one or more sensors configured to collect data representative of a cognitive state of a user while the user performs one or more tasks while interacting with the system; and
a storage unit configured to concurrently record details of the one or more tasks and actions of the user while performing the one or more tasks;
wherein analysis of the data representative of the cognitive state and the recorded details of the one or more tasks and actions of the user is performed, the analysis comprising correlating the data with the recorded details to determine a metric representative of an amount of cognitive state change induced by the system in the user.

10. The system of claim 9, wherein the collecting data and the concurrently recording details for a plurality of users interacting with the system is repeated, and a statistical measure of cognitive state induced by the system on a representative user is generated.

11. The system of claim 10, wherein the statistical measure of cognitive state induced by the system with a second statistical measure of cognitive state induced by a second system is compared, and the first system is ranked as superior to the second system when the statistical measure of cognitive state induced by the system is lower than the second statistical measure of cognitive state induced by the second system.

12. The system of claim 11, wherein the metric representative of the amount of cognitive state change induced by the system in the user is compared with the statistical measure of cognitive state induced by the system on the representative user, and the user is identified as a candidate for additional training when the metric representative of the amount of cognitive state change induced by the system in the user is higher than the statistical measure of cognitive state induced by the system on the representative user by a statistical significant threshold.

13. The system of claim 9, wherein the concurrently recording details of the one or more tasks and actions further comprises temporally correlating the data representative of a cognitive state of the user with a specific task or action of the one or more tasks and actions.

14. The system of claim 9, wherein the data representative of a cognitive state of a user comprises fatigue level, eye movement data, eyelid data, heart rate, respiration rate, electroencephalography (EEG) data, Galvanic Skin Response, functional near-infrared (fNIR) data, electromyography (EMG) data, head position data, head rotation data, emotion, excitement level, Facial Action Coding System (FACS) data, pupillometry, eye tracking data, or cognitive workload data.

15. The system of claim 9, wherein the task comprises a memory training task, a flight training task, a flight simulation task, a virtual surgical task, a virtual driving task, a cognitive assessment task, a cognitive aptitude task, command and control task, air-traffic control task, security monitoring task, vigilance task, a skill aptitude task, or a data entry task.

16. The system of any of claim 9, wherein the cognitive state comprises fatigue level, level of distress, level of excitation, emotion, anxiety level, cognitive overload, cognitive underload, distraction, confusion, level of boredom, a level of tunnel vision, a level of attention, level of stress, level of dementia, level of aptitude, or level of relaxation.

17. An apparatus for determining cognitive state of a user in association with a task, the apparatus comprising:
one or more programmable processors configured to perform operations comprising:
receiving, from one or more sensors data representative of a cognitive state of a user while the user performs one or more tasks while interacting with the system;
concurrently recording details of the one or more tasks and actions of the user while performing the one or more tasks; and
analyzing the data representative of the cognitive state and the recorded details of the one or more tasks and/or actions of the user, the analyzing comprising correlating the data with the recorded details to determine a metric representative of an amount of cognitive state change induced by the system in the user.

18. (canceled)

19. (canceled)

20. The apparatus of claim 17, wherein the data representative of a cognitive state comprises one or more of fatigue level, eye movement data, eyelid data, heart rate, respiration rate, electroencephalography (EEG) data, Galvanic Skin Response, functional near-infrared (fNIR) data, electromyography (EMG) data, head position data, head rotation data, emotion, excitement level, Facial Action Coding System (FACS) data, pupillometry, eye tracking data, or cognitive workload data.

21. (canceled)

22. The apparatus of claim 25, wherein the one or more sensors further include a calibration unit for calibrating the one or more sensors to the user.

23. (canceled)

24. (canceled)

25. The apparatus of claim 17, further comprising the one or more sensors.
